(19) United States
(12) Patent Application Publication
(10) Pub. No.: US 2009/0306169 A1
(43) Pub. Date: Dec. 10, 2009

(54) Use of Fused Pyrrole Carboxylic Acids for the Treatment of Neurodegenerative and Psychiatric Diseases and D-Amino Acid Oxidase Inhibitors

(76) Inventors: Philip E. Brandish, North Wales, PA (US); Timothy Sparey, London (GB); Alister Campbell, London (GB); Andrew Pike, Herts (GB); Nicholas Brandon, Princeton, NJ (US); Wei Zheng, Rahway, NJ (US)

Correspondence Address: MERCK AND CO., INC, P O BOX 2000, RAHWAY, NJ, 07065-0907, US

(21) Appl. No.: 11/992,913

(22) PCT Filed: Oct. 5, 2006
(86) PCT No.: PCT/GB2006/050317

§ 371 Date: Feb. 11, 2009

(30) Foreign Application Priority Data
Oct. 6, 2005 (GB) 0520316.1
Feb. 13, 2006 (GB) 0602783.3

(51) Int. Cl. A61K 31/407; C07D 491/044; C07D 495/04; A61K 31/381

(52) U.S. Cl. 514/412; 548/453; 549/50; 514/443

(57) ABSTRACT

The present invention provides the use of fused pyrrole carboxylic acids of formula (I) for the manufacture of a medicament to inhibit D-amino acid oxidase, particularly for the treatment of neurodegenerative and psychiatric disorders or diseases; certain compounds of formula I being novel, pharmaceutical compositions containing them, their use in medicine and methods of treatment using them are also disclosed.

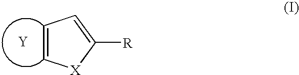

The present invention relates to the use of fused pyrrole carboxylic acids for the treatment of neurodegenerative and psychiatric diseases and disorders, either as a monotherapy or in combination with a further agent useful for the treatment of such diseases and disorders.

PCT patent application WO 03/039540 discloses that compounds of the formula (0):

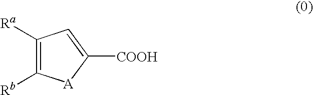

wherein A is oxygen or NH; R^{a} is hydrogen or lower alkyl; R^{b} is hydrogen or lower alkyl; or R^{a} and R^{b} form a six-membered ring, optionally substituted with halogen and/or hydroxyl; are D-amino acid oxidase inhibitors useful for improving learning and memory.

N-methyl-D-aspartate (NMDA)glutamate receptors are expressed at excitatory synapses throughout the central nervous system (CNS). These receptors mediate a wide range of brain processes, including synaptic plasticity associated with certain forms of memory formation and learning. NMDA-glutamate receptors require binding of two agonists to effect neurotransmission. One of these agents is the excitatory amino acid L-glutamate, while the second agonist is thought to be D-serine. In animals D-serine is synthesized from L-serine by serine racemase and degraded to its corresponding keto acid by D-amino acid oxidase. Together, serine racemase and D-amino acid oxidase are thought to play a crucial role in modulating NMDA receptor mediated neurotransmission by regulating CNS concentrations of D-serine. D-amino acid oxidase inhibitors may also modulate other D-amino acid oxidase substrates providing therapeutic activity independent of NMDA receptor activation.

It has now been discovered that a further group of fused pyrrole carboxylic acids have activity as D-amino acid oxidase inhibitors and are useful in the treatment of neurodegenerative and psychiatric disorders and diseases.

Accordingly, in a first aspect, the present invention is directed to the use of a compound of the formula (I):

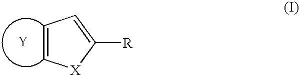

wherein R is a carboxylic acid or a salt, ester, anhydride or amide thereof or a hydroxamic acid or a salt thereof, wherein X is oxygen, sulphur, or NR¹ wherein R¹ is hydrogen, C₁₋₆alkylcarbonyl optionally substituted by one or two amino groups, or R¹ is a group S(O)ₙ R² wherein R² is amino or C₁₋₆alkyl optionally substituted by phenyl and n is 0, 1 or 2, or R¹ is C₁₋₆alkyl optionally substituted by halo or hydroxyl or R¹ is (CH₂)ₘAr, wherein m is 0, 1 or 2 and Ar is as hereinafter defined, which in turn may be substituted by halo, hydroxyl, S(O)ₙ R², wherein R² is a group as hereinbefore defined, or Ar may be substituted by C₁₋₆alkyl, C₁₋₆alkoxy or fluoro-substituted C₁₋₆alkyl or C₁₋₆alkoxy and Y is a five-membered heteroaromatic ring containing at least one hetero atom selected from oxygen, nitrogen and sulphur, which ring may be substituted by one or two substituents which are independently selected from S(O)ₙ R¹, wherein R¹ is amino or C₁₋₆alkyl optionally substituted by phenyl and n is 0, 1 or 2; hydroxyl, halo, amino optionally substituted by one or two C₁₋₆alkyl groups or C₁₋₆alkyl optionally substituted by hydroxyl, halo or amino optionally substituted by one or two C₁₋₆alkyl groups; or the ring Y is optionally substituted by (CH₂)m¹Ar, wherein m¹ is 0, 1 or 2 and Ar is as hereinafter defined, which in turn may be substituted by halo, hydroxyl, amino optionally substituted by one or two C₁₋₆alkyl groups, or Ar is substituted by C₁₋₆alkyl optionally substituted by hydroxyl, halo or amino optionally substituted by one or two C₁₋₆alkyl groups or Ar is optionally substituted by S(O)ₙR₂, wherein R² is amino or C₁₋₆alkyl optionally substituted by phenyl and n is 0, 1 or 2, for the manufacture of a medicament for inhibiting D-amino acid oxidase.

In a further aspect the present invention provides compounds of the formula (I):

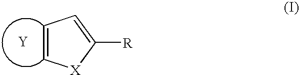

wherein R is a carboxylic acid or a salt, ester, or anhydride thereof or R is a hydroxamic acid or a salt thereof, wherein X is oxygen, sulphur, or NR¹ wherein R¹ is hydrogen, C₁₋₆alkylcarbonyl optionally substituted by one or two amino groups, or R¹ is a group S(O)ₙR² wherein R² is amino or C₁₋₆alkyl optionally substituted by phenyl and n is 0, 1 or 2, or R¹ is C₁₋₆alkyl optionally substituted by halo or hydroxyl or R¹ is (CH₂) , wherein m is 0, 1 or 2 and Ar is as hereinafter defined, which in turn may be substituted by halo, hydroxyl, S(O)ₙR², wherein R² is a group as hereinbefore defined, or Ar may be substituted by C₁₋₆alkyl, C₁₋₆alkoxy or fluoro-substituted C₁₋₆alkyl or C₁₋₆alkoxy and Y is a five-membered heteroaromatic ring containing at least one hetero atom selected from oxygen, nitrogen and sulphur, which ring may be substituted by one or two substituents which are independently selected from S(O)ₙ R¹, wherein R¹ is amino or C₁₋₆alkyl optionally substituted by phenyl and n is 0, 1 or 2; hydroxyl, halo, amino optionally substituted by one or two C₁₋₆alkyl groups or C₁₋₆alkyl optionally substituted by hydroxyl, halo or amino optionally substituted by one or two C₁₋₆alkyl groups; or the ring Y is optionally substituted by (CH₂)ₘ¹Ar, wherein m¹ is 0, 1 or 2 and Ar is as hereinafter defined, which in turn may be substituted by halo, hydroxyl, amino optionally substituted by one or two C₁₋₆alkyl groups, or Ar is substituted by C₁₋₆alkyl optionally substituted by hydroxyl, halo or amino optionally substituted by one or two C₁₋₆alkyl groups or Ar is optionally substituted by S(O)ₙR², wherein R² is amino or C₁₋₆alkyl optionally substituted by phenyl and n is 0, 1 or 2; for use in medicine.

Ar is a five- or six-membered aromatic ring which may be carbocyclic or heterocyclic. Preferred rings Ar include phenyl, pyridyl and thiazolyl, in particular phenyl.

Preferably, Y is a five-membered heteroaromatic ring containing one oxygen or sulphur atom. Suitable substituents for the ring include halo, hydroxyl, methyl or trifluoromethyl. Preferably, the ring is unsubstituted.

A preferred group of compounds of the formula (1) is that of formula (H):

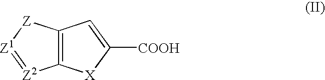

or a salt, ester or anhydride thereof, wherein X is oxygen, sulphur, or a group NR¹ wherein R¹ is hydrogen, C₁₋₆alkyl optionally substituted by halo, hydroxyl or (CH₂)ₘAr, wherein m is 0, 1 or 2 and Ar is as hereinafter defined, which group Ar in turn may be substituted by halo, hydroxyl, S(O)ₙR², wherein R² is amino, C₁₋₆alkyl optionally substituted by phenyl and n is 0, 1 or 2; or Ar may be substituted by C₁₋₆alkyl, C₁₋₆alkoxy or fluoro-substituted C₁₋₆alkyl or C₁₋₆alkoxy; Z is an oxygen or sulphur atom or a group NR¹ as hereinbefore defined; one of Z¹ and Z² is CR³ or N, and the other is CR³, wherein R³ is hydrogen or halo. Preferably Z is oxygen or sulphur. Most suitably R³ is hydrogen, chloro or bromo. Preferably, R³ is hydrogen.

A further preferred of compounds of the formula (I) is that of formula (III):

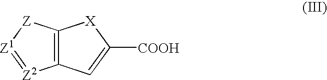

or a salt, ester or anhydride thereof, wherein X is oxygen, sulphur, or a group NR¹ wherein R¹ is hydrogen, C₁₋₆alkyl optionally substituted by halo, hydroxyl or R¹ is a group (CH₂)ₘAr, wherein m is 0, 1 or 2 and Ar is as herein defined, which group Ar in turn may be substituted by halo, hydroxyl, S(O)ₙR², wherein R² is amino, C₁₋₆alkyl optionally substituted by phenyl and n is 0, 1 or 2; or Ar may be substituted by C₁₋₆alkyl, C₁₋₆-alkoxy or fluoro-substituted C₁₋₆alkyl or C₁₋₆alkoxy; Z is an oxygen or sulphur atom or a group NR¹ as hereinbefore defined; one of Z¹ and Z² is CR³ or N and the other is CR³, wherein R³ is hydrogen or halo. Suitably Z is oxygen or sulphur and preferably Z is sulphur. Most suitably Z¹ is CH, N or Chalo¹ wherein halo¹ is chloro or bromo. Preferably, R³ is hydrogen.

Preferred compounds of the formula (I) include:

4H-Thieno[3,2-b]pyrrole-5-carboxylic acid;
6H-Thieno[2,3-b]pyrrole-5-carboxylic acid;
3-Bromo-4H-furo[3,2-b]pyrrole-5-carboxylic acid;
4H-Furo[3,2-b]pyrrole-5-carboxylic acid; and
3-Chloro4H-furo[3,2-b]pyrrole-5carboxylic acid.

As appreciated by those of skill in the art, halo or halogen as used herein are intended to include fluoro, chloro, bromo and iodo. Similarly, C₁₋₆, as in, C₁₋₆alkyl is defined to identify the group as having 1, 2, 3, 4, 5 or 6 carbons in a linear or branched arrangement, such that C₁₋₆alkyl specifically includes methyl, ethyl, n-propyl, iso-propyl, n-butyl, iso-butyl, tert-butyl, pentyl and hexyl.

As used herein, aromatic heterocyclic moieties (i.e. “heteroaryl”) include furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, and N-oxides thereof,

Specific embodiments of the present invention include a compound which is selected from the group consisting of the subject compounds of the examples herein and salts, esters, anhydrides and amides thereof and, where appropriate, individual enantiomers and diastereomers thereof.

Certain compounds of the formula (I) are novel. Thus, in a further aspect, the present invention provides a novel compound of the formula (I). Preferred novel compounds include:

4H-Furo[3,2-b]pyrrole-5-carboxylic acid,
6H-Furo[2,3-b]pyrrole-5-carboxylic acid,
3-Bromo-4H-furo[3,2-b]pyrrole-5-carboxylic acid and
2-Chloro-4H-furo[3,2-b]pyrrole-5-carboxylic acid.

The compounds of the present invention may contain one or more chiral centers depending on the nature of any substituents present, and can thus occur as racemates and racemic mixtures, single enantiomers, diastereomeric mixtures and individual diastereomers. It is intended that all of the possible optical isomers and diastereomers in mixtures and as pure or partially purified compounds are included within the ambit of this invention. The present invention is meant to comprehend all such isomeric forms of these compounds. Formula I shows the structure of the class of compounds without preferred stereochemistry.

The salts of the present invention are preferably pharmaceutically acceptable. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfiric, fumaric, and tartaric acids. It will be understood that, as used herein, references to the compounds of the present invention are meant to also include the pharmaceutically acceptable salts. In addition, salts of the compounds of the formula (I) may be valuable intermediates in preparation of other compounds of the formula (I).

The esters of the present invention are preferably pharmaceutically acceptable esters that are cleavable in-vivo to the parent carboxylic acids of the formula (I). However, esters that are not pharmaceutically acceptable may be valuable intermediates in the preparation of other compounds of the formula (I) and hence comprise a further aspect of the present invention. Preferred esters of the present invention include C₁₋₆alkyl esters, such as unbranched C₁₋₄alkyl esters.

Hydroxamic acids of the compound of the formula (1) include those of the formula (IV):

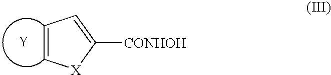

The amides of the present invention are preferably pharmaceutically acceptable amides that are cleavable in-vivo to the parent carboxylic acids of the formula (I).

The present invention also includes within its scope N-oxides of the compounds of formula (I) above. In general, such N-oxides may be formed on any available nitrogen atom, and preferably on any one of X, Z or Z¹ where they represent a nitrogen atom. The N-oxides may be formed by conventional means, such as reacting the compound of formula (I) with oxone in the presence of wet alumina.

In a further aspect, the present invention provides pharmaceutical compositions which comprise a pharmaceutically effective amount of a compound of the formula (I) in admixture with a pharmaceutically acceptable carrier.

The subject compounds are useful in a method of inhibiting D-amino acid oxidase activity in a patient such as a mammal in need of such inhibition comprising the administration of an effective amount of the compound. The present invention is directed to the use of the compounds disclosed herein as inhibitors of D-amino acid oxidase. In addition to primates, especially humans, a variety of other mammals can be treated according to the method of the present invention.

The present invention is further directed to a method for the manufacture of a medicament for inhibiting D-amino acid oxidase activity in humans and animals which comprises combining a compound of the present invention with a pharmaceutical carrier or diluent.

The subject treated in the present methods is generally a mammal, preferably a human being, male or female, in whom inhibition of D-amino acid oxidase activity is desired. The term “therapeutically effective amount” means the amount of the subject compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with such disorders with an effective amount of the compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy to retard the progression or reduce the risk of the noted conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibiting D-amino acid oxidase activity may be demonstrated by methodology known in the art.

The compounds of the present invention have utility in treating a variety of neurological and psychiatric disorders associated with glutamatergic neurotransmission dysfunction, including one or more of the following conditions or diseases: schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anaesthetics, amphetamine and other psychostimulants and cocaine) psychosispsychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both the positive and the negative symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder, tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD) and conduct disorder, NMDA receptor-related disorders such as autism, depression, benign forgetfulness, childhood learning disorders and closed head injury, movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors; dyskinesias [including tremor (such as rest tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalized myoclonus and focal myoclonus), tics (including simple tics, complex tics and symptomatic tics),and dystonia (including generalized dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)]; urinary incontinence; neuronal damage including ocular damage, retinopathy or macular degeneration of the eye, tinnitus, hearing impairment and loss, and brain edema; emesis; and sleep disorders including insomnia and narcolepsy.

Of the disorders above, the treatment of schizophrenia, bipolar disorder, depression including unipolar depression, seasonal depression and post-partum depression, premenstrual syndrome (PMS) and premenstrual dysphoric disorder (PDD), learning disorders, pervasive developmental disorder including autistic disorder, attention disorders including Attention-Deficit/Hyperactivity Disorder, autism, tic disorders including Tourette's disorder, anxiety disorders including phobia and post traumatic stress disorder, cognitive disorders associated with dementia, AIDS dementia, Alzheimer's, Parkinson's, Huntington's disease, spasticity, myoclonus, muscle spasm, tinnitus and hearing impairment and loss are of particular importance.

In a specific embodiment, the present invention provides a method for treating cognitive disorders, comprising: administering to a patient in need thereof an effective amount of a compound of the present invention. Particular cognitive disorders are dementia, delirium, amnestic disorders and age-related cognitive decline. At present, the text revision of the fourth edition of the Diagnostic and Statistical Manual of Mental Disorders (DSM-IV-TR) (2000, American Psychiatric Association, Washington D.C.) provides a diagnostic tool that includes cognitive disorders including dementia, delirium, amnestic disorders and age-related cognitive decline. As used herein, the term “cognitive disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In another specific embodiment, the present invention provides a method for treating anxiety disorders, comprising administering to a patient in need thereof an effective amount of a compound of the present invention. Particular anxiety disorders are generalized anxiety disorder, obsessive-compulsive disorder and panic attack. At present, the text revision of the fourth edition of the Diagnostic and Statistical Manual of Mental Disorders (DSM-IV-TR) (2000, American Psychiatric Association, Washington D.C.) provides a diagnostic tool that includes anxiety disorders are generalized anxiety disorder, obsessive-compulsive disorder and panic attack. As used herein, the term “anxiety disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “anxiety disorders” is intended to include like disorders that are described in other diagnostic sources.

In another specific embodiment, the present invention provides a method for treating schizophrenia or psychosis comprising: administering to a patient in need thereof an effective amount of a compound of the present invention. Particular schizophrenia or psychosis pathologies are paranoid, disorganized, catatonic or undifferentiated schizophrenia and substance-induced psychotic disorder. At present, the text revision of the fourth edition of the Diagnostic and Statistical Manual of Mental Disorders (DSM-IV-TR) (2000, American Psychiatric Association, Washington DC) provides a diagnostic tool that includes paranoid, disorganized, catatonic or undifferentiated schizophrenia and substance-induced psychotic disorder. As used herein, the term “schizophrenia or psychosis” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

In another specific embodiment, the present invention provides a method for treating substance-related disorders and addictive behaviors, comprising: administering to a patient in need thereof an effective amount of a compound of the present invention. Particular substance-related disorders and addictive behaviors are persisting dementia, persisting amnestic disorder, psychotic disorder or anxiety disorder induced by substance abuse; and tolerance of, dependence on or withdrawal from substances of abuse. At present, the text revision of the fourth edition of the Diagnostic and Statistical Manual of Mental Disorders (DSM-IV-TR) (2000, American Psychiatric Association, Washington D.C.) provides a diagnostic tool that includes persisting dementia, persisting amnestic disorder, psychotic disorder or anxiety disorder induced by substance abuse; and tolerance of, dependence on or withdrawal from substances of abuse. As used herein, the term “substance-related disorders and addictive behaviors” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “substance-related disorders and addictive behaviors” is intended to include like disorders that are described in other diagnostic sources.

The compounds of the present invention will be of use in the prevention or treatment of diseases and conditions in which pain and/or inflammation predominates, including chronic and acute pain conditions. Such conditions include rheumatoid arthritis; osteoarthritis; post-surgical pain; musculo-skeletal pain, particularly after trauma; spinal pain; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, for example, odontalgia, abdominal pain, gynaecological pain, for example, dysmenorrhoea, pain associated with cystitis and labor pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; itching conditions including pruritis, itch due to hemodialysis, and contact dermatitis; pain (as well as broncho-constriction and inflammation) due to exposure (e.g. via ingestion, inhalation, or eye contact) of mucous membranes to capsaicin and related irritants such as tear gas, hot peppers or pepper spray, neuropathic pain conditions such as diabetic neuropathy, chemotherapy-induced neuropathy and post-herpetic neuralgia; “non-painful” neuropathies; complex regional pain syndromes; pain associated with carcinoma, often referred to as cancer pain; central nervous system pain, such as pain due to spinal cord or brain stem damage, low back pain, sciatica and ankylosing spondylitis; gout; scar pain; irritable bowel syndrome; inflammatory bowel disease; urinary incontinence including bladder detrusor hyper-reflexia and bladder hypersensitivity; respiratory diseases including chronic obstructive pulmonary disease (COPD), chronic bronchitis, cystic fibrosis and asthma; autoimmune diseases; and immunodeficiency disorders.

In another specific embodiment, the present invention provides a method for treating pain, comprising: administering to a patient in need thereof an effective amount of a compound of the formula (I) or a composition comprising a compound of formula (I). Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

According to a further or alternative aspect, the present invention provides a compound of formula (I) for use in the manufacture of a medicament for the treatment or prevention of a disease or condition in which pain and/or inflammation predominates.

In another specific embodiment, the present invention provides a method for treating obesity or eating disorders associated with excessive food intake and complications associated therewith, comprising: administering to a patient in need thereof an effective amount of a compound of the present invention. At present, obesity is included in the tenth edition of the International Classification of Diseases and Related Health Problems (ICD-10) (1992 World Health Organization) as a general medical condition. The text revision of the fourth edition of the Diagnostic and Statistical Manual of Mental Disorders (DSM-WV-TR) (2000, American Psychiatric Association, Washington D.C.) provides a diagnostic tool that includes obesity in the presence of psychological factors affecting medical condition. As used herein, the term “obesity or eating disorders associated with excessive food intake” includes treatment of those medical conditions and disorders described in ICD-10 and DSM-IV-TR. The skilled artisan will recognize that there are alternative nomenclatures, nosologies and classification systems for general medical conditions, and that these systems evolve with medical and scientific progress. Thus the term “obesity or eating disorders associated with excessive food intake” is intended to include like conditions and disorders that are described in other diagnostic sources.

The subject compounds are further useful in a method for the prevention, treatment, control, amelioration, or reducation of risk of the diseases, disorders and conditions noted herein.

The subject compounds are further useful in a method for the prevention, treatment, control, amelioration, or reduction of risk of the aforementioned diseases, disorders and conditions in combination with other agents, including an inhibitor of glycine transporter GlyT1 activity

The compounds of the present invention may be used in combination with one or more other drugs in the treatment, prevention, control, amelioration, or reduction of risk of diseases or conditions for which compounds of the present invention or the other drugs may have utility, where the combination of the drugs together are safer or more effective than either drug alone. Such other drug(s) may be administered, by a route and in an amount commonly used therefore, contemporaneously or sequentially with a compound of the present invention. When a compound of the present invention is used contemporaneously with one or more other drugs, a pharmaceutical composition in unit dosage form containing such other drugs and the compound of the present invention is preferred. However, the combination therapy may also include therapies in which the compound of the present invention and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of the present invention.

The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefore, contemporaneously or sequentially with a compound of the present invention. When a compound of the present invention is used contemporaneously with one or more other drugs, a pharmaceutical composition containing such other drugs in addition to the compound of the present invention is preferred Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient Generally, an effective dose of each will be used. Thus, for example, when a compound of the present invention is combined with another agent, the weight ratio of the compound of the present invention to the other agent will generally range from about 1000: 1 to about 1: 1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

Accordingly, the subject compounds may be used alone or in combination with other agents which are known to be beneficial in the subject indications or other drugs that affect receptors or enzymes that either increase the efficacy, safety, convenience, or reduce unwanted side effects or toxicity of the compounds of the present invention. The subject compound and the other agent may be coadministered, either in concomitant therapy or in a fixed combination.

In one embodiment, the subject compound may be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies.

In another embodiment, the subject compound may be employed in combination with sedatives, hypnotics, anxiolytics, antipsychotics, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, zolpidem, and salts thereof, and combinations thereof, and the like, or the subject compound may be administered in conjunction with the use of physical methods such as with light therapy or electrical stimulation.

In another embodiment, the subject compound may be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexol) hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In another embodiment, the subject compound may be employed in combination with a compound from the phenothiazine, thioxanthene, heterocyclic dibenzazepine, butyrophenone, diphenylbutylpiperidine and indolone classes of neuroleptic agent Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound may be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In another embodiment, the subject compound may be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase ), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In another embodiment, the subject compound may be employed in combination with a compound useful in the treatment of pain, for example an NSAID such as ibuprofen, an antinociceptive agent such as an NR2B antagonist, a COX2 inhibitor such as ARCOXIA or a sodium channel blocker.

The compounds of the present may also be employed in combination with D-amino acids or suitable derivatives thereof such as D-phenylalanine, parafluoro-D-phenyl alanine, D-(N-trifluoroacetyl-4fluorophenylalanine), D-leucine, D-alanine, D-cycloserine and Dserine or D/L mixtures thereof.

Preferred combinations of the present invention include compounds of the formula (1) in combination with D-serine, clozapine, haloperidole, olanzapine, or risperidone.

The compounds of the present invention may be administered by oral, parenteral (e.g., intramuscular, intraperitoneal, intravenous, ICV, intracistemal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. The tablets may be uncoated or they may be coated by known techniques to delay disintegration and absorption in the gastrointestinal tract and thereby provide a sustained action over a longer period or may be tablets that disperse when added to water. Compositions for oral use may also be presented as hard gelatin capsules wherein the active ingredients are mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions, oily suspensions, dispersible powders or granules, oil-in-water emulsions, and sterile injectable aqueous or oleagenous suspension may be prepared by standard methods known in the art.

In the treatment of conditions which require inhibition of aramino acid oxidase activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, preferably once or twice per day. This dosage regimen may be adjusted to provide the optimal therapeutic response. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials and the requisite intermediates are in some cases commercially available, or can be prepared according to literature procedures or as illustrated herein.

The compounds of this invention may be prepared by employing methods well known to those skilled in the art for preparing analogous compounds, for example using the reactions as shown in the following schemes, in addition to other standard manipulations that are known in the literature or exemplified in the experimental procedures. Substituent numbering as shown in the schemes does not necessarily correlate to that used in the claims and often, for clarity, a single substituent is shown attached to the compound where multiple substituents are allowed under the definitions hereinabove. Reactions used to generate the compounds of this invention are prepared by employing reactions as shown in the schemes and examples herein, in addition to other standard manipulations such as ester hydrolysis, cleavage of protecting groups, etc., as may be known in the literature or exemplified in the experimental procedures.

Accordingly, in a further aspect, the present invention provides a process for the preparation of a compound of the formula (1) as hereinbefore defined, which process comprises the cyclisation of an ester of a compound of the formula (V):

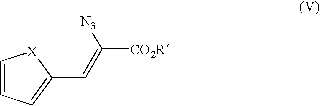

wherein CO₂R′ is an ester group as hereinbefore described. The cyclisation is conveniently carried out in a non-reactive solvent, for example a hydrocarbon such as xylene, at an elevated temperature, for example between 50 and 150° C. and conveniently at reflux.

The independent syntheses of diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

In some cases the final product may be further modified, for example, by manipulation of substituents. These manipulations may include, but are not limited to, reduction, oxidation, alkylation, acylation, and deesterification/hydrolysis reactions which are commonly known to those skilled in the art. Certain compounds of the formula (1) may therefore be useful as intermediates in preparation of other compounds of the formula (1). In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### EXAMPLE 1

4H-Furo[3,2-b]pyrrole-5-carboxylic Acid Ethyl Ester
a) 2-Azido-3-furan-2-yl-acrylic Acid Ethyl Ester
To a solution of sodium ethoxide (53 g) in EtOH (1 litre) at 0° C. was added 2-furancarboxaldehyde (62 g) and ethyl azidoacetate (100 g). This reaction mixture was stirred at this temperature for 4 h until the aldehyde was completely consumed. Then the resulting mixture was poured into saturated aqueous NH₄Cl and extracted with ether once. A second extraction produced a less pure sample of product The first extract was evaporated and purified by column chromatography on silica eluting with DCM/hexane to give 50 g of product (37%). ¹H NMR δ (ppm)(CDCl3): 7.49 (1 H, d, J=1.5 Hz), 7.10 (1 H. t, J=1.8 Hz), 6.87 (1 H s), 6.52 (1 H, m), 4.34 (2 H. q, J=7.1 Hz), 1.38 (4 H, t, J=7.2 Hz).
b) 4H-Furo[3,2-b]pyrrole-5-carboxylic Acid Ethyl Ester
2-Azido-3-furan-2-yl-acrylic acid ethyl ester (50 g) was added to refluxing xylene (100 ml), and stirred for 5 min. The solvent was concentrated under reduced pressure, and the residue was purified by column chromatography eluting with DCM/hexane to provide the product in good yield (37 g, 86%). ¹H NMR δ (ppm)(CDCl₃): 8.81 (1 H, s), 7.51 (1 H, d, J=2.2 Hz), 6.80 (1 H, s), 6.45 (1 H, d, J=1.6 Hz), 4.35 (2 H, q, J=7.1 Hz), 1.38 (3 H, t, J=7.1 Hz).

### EXAMPLE 2

4H-Furo[3,2-b]pyrrole-5-carboxylic Acid
4H-Furo[3,2-b]pyrrole-5-carboxylic acid ethyl ester was suspended in water and potassium hydroxide (2 equivalents) was added. The mixture was heated to reflux for 30 minutes, cooled to 10° C. and acidified with 5N HCl. The solid was filtered off, washed with water and dried to provide the product as a light beige solid (30 g, 97%). 1H NMR 6 (ppm)(DMSO): 12.33 (1 H, s), 11.48 (1 H, s), 7.75 (1 H, d, J=2.1 Hz), 6.68 (1 H, s), 6.58 (1 H, d, J=1.5 Hz).

### EXAMPLES 3-10

The following compounds were synthesized using the procedure described above, except for using the appropriate aldehyde:
3) 4H-Thieno[3,2-b]pyrrole-5-carboxylic Acid
1H NMR δ (ppm)(CD₃OD): 7.35 (1 H, d, ), 7.06 (1 H, s), 6.96 (1 H, d, J=2.1 Hz); API-ES: 168 (MH+)
4) 6H-Thieno[2,3-b]pyrrole-5-carboxylic Acid
1H NMR δ (ppm)(DMSO): 12.50 (1 H, s), 12.08 (1 H, s), 7.09 (1 H, d, J=5.5 Hz), 7.01 (1 H, d, J=5.5 Hz), 6.94 (1 H, s).
5) 3-Bromo-4H-furo[3.2-b]pyrrole-5-carboxylic Acid
1H NMR δ (ppm)(CD₃OD): 7.49 (1 H, s), 6.56 (1 H, s); API-ES (+ve): 230 (MH+)
6) Thieno[2,3-b]furan-5-carboxylic Acid
1H NMR δ (ppm)(CD₃OD): 7.82 (1 H, s), 7.72 (1 H, s), 6.83 (1 H, s)
7) 2-Chloro-4H-thieno[3,2-b]pyrrole-5-carboxylic Acid
1H NMR δ (ppm)(CD₃OD): 6.99 (1 H, s), 6.96 (1 H, s); API-ES (+ve): 202 (MH+)
8) 2-Methyl-4H-thieno[3,2-b]pyrrole-5-carboxylic Acid
1H NMR δ (ppm)(CD₃OD): 6.97 (1 H, s), 6.68 (1 H, s), 2.52 (3 H, s); API-ES (−ve): 180 (M-H)
9) 2-Chloro-4H-furo[3,2-b]pyrrole-5-carboxylic Acid
1H NMR δ (ppm)(CD₃OD): 6.69 (1 H, s), 6.45 (1 H, s); API-ES (+ve): 188 (MH+)
10) 2-(2-Trifluoromethoxy-phenyl)-4H-furo[3,2-b]pyrrole-5-carboxylic Acid
3-Bromo-4H-furo[3,2-b]pyrrole-5-carboxylic acid ethyl ester and 2-trifluoromethoxy)phenyl boronic acid (1.2 eq) were dissolved in DMF and then saturated Na₂CO₃ (2.5 eq) was added. The resulting mixture was de-gassed with N₂ three times, and stirred for 30 min. Then Pd(dppf)Cl₂ was added under N₂ and the reaction stirred at 100° C. overnight. The solvent was removed under reduced pressure and the residue was re-dissolved in DCM, and washed with water and brine. Purification by PTLC gave the ester which was saponified as described above. 1H NMR δ (ppm)(CD₃OD): 8.01 (1 H, d, J=9.6Hz), 7.41 (3H, m), 6.87 (1 H, s), 6.76 (1 H, s); API-ES (−ve): 310 (M-H)

### EXAMPLE 11

Ethyl 3-bromo-4H-thieno[3,2-b]pyrrole-5-carboxylate

### Step 1.

Ethyl azidoacetate (1.014 g, 7.85 mmol) was added to 10 ml EtOH and cooled to −40° C. on an acetone/cardice bath before addition of 21% by wt sodium ethoxide solution in EtOH (3.16 ml, 8.37 mmol) dropwise from a syringe. The resultant solution turned brown in color and became thick.4-bromothiophene-2-carboxaldehyde (1 g, 5.23 mmol) was then slowly added to this solution over 20 minutes and the mixture stirred at −40° C. for 2 hours then allowed to slowly warm to room temp over 3 hours. After this time 200 ml Et₂O was added along with 100 ml of sat NH₄Cl aqueous solution. The organics were separated and dried (MgSO₄), filtered and the solvent was evaporated under reduced pressure. The residue was dryloaded onto silica and purified by column chromatography on silica gel Biotage 25M, eluting with a linear gradient of EtOAc/isohexane to give the desired ethyl (2Z)-2-azido-3-(4-bromo-2-thienyl)acrylate as a yellow solid (705 mg, 38% yield, 85% purity).

### Step 2.

Ethyl (2Z-2-azido-3-(4-bromo-2-thienyl)acrylate (705 mg, 1.983 mmol) was dissolved in Xylene (40 ml) and heated to 140° C. for 2 hours. The xylene was removed under reduced pressure and the residue was purified by column chromatography on silica gel Biotage 25S, eluting with EtOAc/isohexane to give ethyl 3-bromo-4H-thieno[3,2-b]pyrrole-5-carboxylate (393 mg, 1.434 mmol, 72.3% yield) as a pale yellow solid.

### EXAMPLE 12

3-bromo-4H-thieno[3,2-b]pyrrole-5-carboxylic Acid
Ethyl 3-bromo-4H-thieno[3,2-b]pyrrole-5-carboxylate (100 mg, 0.36 mmol) was dissolved in a mixture of Water (10 ml) and Methanol (10.0 ml) then treated with potassium hydroxide (22 mg, 0.55 mmol) before being heated to 85° C. for 3 hours. The mixture was cooled, and the methanol was removed in vacuo. The aqueous was then acidified with 20 ml of 1 M HCl solution, resulting in the formation of a precipitate. The mixture was filtered, washing with water giving 3-bromo-4H-thieno[3,2-b]pyrrole-5-carboxylic acid (74 mg, 0.3 mmol, 84% yield) as a beige solid. 1H NMR (500 MHz, CDCl3): δ 9.03 (1H, s), 7.26 (1H, s), 7.22 (1H, s), 7.15 (1H, d, J 1.7).

### EXAMPLE 13

Ethyl 3-chloro-4H-thieno[3,2-b]-pyrrole-5-carboxylate
Ethyl 3-bromo-4H-thieno[3,2-b]pyrrole-5-carboxylate (100 mg, 0.36 mmol) was dissolved dry DMF (15 ml) and treated with CuCl (71 mg, 0.72 mmol) and heated to reflux for 16 hours. The mixture was then cooled diluted with water (100 ml) and extracted with 2×150 ml EtOAc, which was then washed with 1×100 ml water and 1×100 ml saturated brine solution. The organics were then separated, dried (MgSO₄) and concentrated in vacuo. The residue was then purified by mass directed preparative LCMS giving ethyl 3-chloro4H-thieno[3,2-b]pyrrole-5-carboxylate (32 mg, 0.13 mmol, 38% yield) as a colorless solid.

### EXAMPLE 14

3-chloro-4H-thieno[3,2-b]pyrrole-5-carboxylic Acid
Ethyl 3-chloro4H-thieno[3,2-b]pyrrole-5-carboxylate (32 mg, 0.13 mmol) was dissolved in a mixture of water (5 ml) and methanol (5 ml) then treated with potassium hydroxide (10 mg, 0.26 mmol) before being heated to 85° C. for 3 hours. The mixture was cooled, and the methanol was removed in vacuo. The aqueous solution was then acidified with 15 ml of 1M HCl solution, resulting in the formation of a precipitate. The mixture was extracted with 50 ml EtOAc which was then washed with 1×50 ml water and 1×50 ml saturated brine solution. The organics were then separated, dried (MgSO₄) and concentrated in vacuo giving 3-bromo-4H-thieno[3,2-b]pyrrole-5-carboxylic acid (22 mg, 0.11 mmol, 92% yield) as a colorless solid. 1H NMR (500 MHz, DMSO): δ 12.74 (1H, s), 12.40 (1H, s), 7.51 (1H, s), 7.09 (1H, s).

### EXAMPLE 15

2-(2-phenylethyl)4H-thieno[3,2-b]pyrrole-5-carboxylic Acid
Ethyl 2-bromo-4H-thieno[3,2-b]pyrrole-5-carboxylate (80 mg, 0.292 mmol) was dissolved in NMP (5 ml) and to it was added phenethyl bromide 0.5M solution in THF (5.84 ml, 2.92 mmol) and bis(tri-tbutylphosphine)palladium(0) (2.98 mg, 5.84 μmol) and the resultant solution heated to 100° C. for 16 hours under an atmosphere of N₂ The mixture was cooled, diluted with ethyl acetate (100 mL), washed with aqueous ammonium chloride (saturated 150 mL), then aqueous sodium chloride (saturated, 150 mL) dried (MgSO₄), filtered and the solvent was evaporated under reduced pressure. The crude residue was suspended in a water (25 ml)/methanol (25 ml) mixture and potassium hydroxide (32.7 mg, 0.584 mmol) was added before the mixture was heated to reflux for 3 hours. After this time the methanol was removed under reduced pressure and the aqueous solution acidified with the addition of 1M HCl solution resulting in the formation of a precipitate which was collected by filtration. The precipitate was then dissolved in 2 ml DMSO and purified by preparative HPLC Reverse phase (C-18), eluting with Acetonitrile/Water+0.1% TFA, to give 2-2-phenylethyl)-4H-thieno[3,2-b]pyrrole-5-carboxylic acid (62.4 mg, 0.230 mmol, 79% yield) as an off-white solid. 1H NMR (500 MHz, DMSO): δ 11.70 (1H, s), 7.31-7.23 (4H, m), 7.17 (1H, t, J 6.7), 6.91 (1H, d, J 9.6), 6.71 (1H, d, J 8.9), 3.12 (2H t, J 7.7), 2.94 (2H, t, J 7.7).

### EXAMPLE 16

Ethyl 2-benzyl-4H-thieno[3,2-b]pyrrole-5carboxylate
Ethyl 2-bromo4H-thieno[3,2-b]pyrrole-5-carboxylate (50 mg, 0.182 mmol) was dissolved in NMP (5 ml) and to it was added benzylzinc bromide 0.5M in THF (3.65 ml, 1.824 mmol) and bis(tri-tbutylphosphine)palladium(0) (1.864 mg, 3.65 μmol) and the resultant solution heated to 100° C. for 16 hours under an atmosphere of N₂. After this time the mixture was cooled, aqueous ammonium chloride (saturated, 150 mL) was added and the mixture was extracted with ethyl acetate (1×200 mL). The organic layer was washed with water (1×150 mL), dried (MgSO₄), filtered and the solvent was evaporated under reduced pressure giving an orange oil. The residue was purified by column chromatography on silica gel Biotage 12M, eluting with 7% EtOAc/isohexane to give ethyl 2-benzyl-4H-thieno[3,2-b]pyrrole-5carboxylate (52 mg, 0.128 mmol, 69.9% yield) as a colorless solid. 1H NMR (400 MHz, CDCl3): δ 9.11 (1H, s), 7.29-7.15 (6H, m), 6.57 (1H, s), 4.32-4.24 (2H, m), 4.08 (2H, s), 1.30-1.26 (3H,

### EXAMPLE 17

2-benzyl-4H-thieno[3,2-b]pyrrole-5-carboxylic Acid
Ethyl 2-benzyl-4H-thieno[3,2-b]pyrrole-5-carboxylate (52 mg, 0.128 mmol) was dissolved in methanol (25 ml) and then water (25.00 ml) before addition of potassium hydroxide (21.47 mg, 0.383 mmol); the mixture was heated to reflux for 4 hours. After this time the reaction was cooled and the methanol was removed in vacuo and the aqueous solution was acidified with 2M aqHCl solution resulting in the formation of a white precipitate. This was isolated by extraction with 100 ml EtOAc, the organic fraction being separated then dried (MgSO₄), filtered and the solvent evaporated under reduced pressure. The residue was purified by preparative HPLC Reverse phase (C-18), eluting with acetonitrile/water+0.1% TFA, to give 2-benzyl-4H-thieno[3,2-b]pyrrole-5-carboxylic acid (23.4 mg, 0.091 mmol, 71.3% yield) as a colorless solid. 1H NMR (500 MHz, DMSO): δ 12.39 (1H, s), 11.76 (1H, s), 7.34-7.28 (4H, m), 7.24-7.22 (1H, m), 6.93 (1H, d, J 1.5), 6.76 (1H, s), 4.15 (2H, s).

### EXAMPLE 18

### a) Cell Based Assay Protocol to Determine Efficacy of D-amino Acid Oxidase Inhibitors

CHO cells stably expressing human D-amino acid oxidase were grown in F12/Ham glutamax medium containing 10% FBS, 1× pen/strep and 1 mg/ml G418. On the day of assay, cells were washed with PBS, harvested and spun at 1000 rpm for 5 mins before resuspending in assay buffer (HBSS containing 1 M CaCl 1M MgCl₂ and 1M Hepes, pH 7.4) at 8.6×10⁵/ml. 35 ul cell suspension was added to 5 ul test compound in a 384 well plate. The assay was initiated by the addition of 10 ul assay buffer containing 2.5% amplex red (Molecular Probes), 6% horse radish peroxidase and 25% 1M D-serine. Plates were incubated for 2 hours at 37° C. and fluorescence (excitation 544 nm, emission 590 nm) read using a Cytofluor plate reader. Compounds of the present invention had activity at below the one micromolar level.

### b) EDID Cognition Assay

EDID has been adapted for use with non-human primates, where it has been shown that selective ED shift deficits similar to those observed in first episode schizophrenia can be induced by excitotoxic lesions of the dorsolateral prefrontal cortex (Dias et al., Behav. Neurosci., 110, 872-886, 1996). More recently, an analogue of this test has been developed (Birrell and Brown, J. Neurosci., 20,4320-4324, 2000), and refined (Barense et al., Learn Mem., 9, 191-201, 2002) for use in rodents. Specifically, this task requires rats to solve a series of discrimination problems parallel to those presented in the CANTAB EDID test by distinguishing which of two pots presented contains food rewards based on two or three non-spatial cue dimensions (odor, digging medium, and/or texture). Birrell and Brown have shown that rats with selective medial prefrontal cortex lesions are impaired at ED shift discriminations, but not at intradimensional (ID) shift or reversal discriminations (IDR or EDR). In addition, sub-chronic PCP administration plus washout period has also been shown to impair performance in this assay, inducing a selective ED shift deficit (Egerton et al., Psychopharmacology, 179, 77-84, 2005).
Adult male Hooded Lister rats (200-350 g at the time of testing) purchased from Charles River were housed together (4 rats per cage) under controlled conditions (12 hours of light starting at 07:30; 21±2° C.; 55±10% humidity) in solid-bottomed cages with woodchip bedding and environmental enrichment (cardboard tubes and/or wooden chew blocks). Rats were given access to food and water ad libitum from Friday afternoon through Sunday morning and for the remainder of the week each rat consumed approximately 11 g of food each per day. Rat weights were closely monitored to ensure that no individual animal dropped below 85% of its free feeding weight during food restriction.
Drugs. Phencyclidine (PCP; Ultrafine Chemicals, Poole, UK) was prepared in normal saline at 5 mg/ml using a salt-base ratio of 1.15.
Rats were administered sub-chronic PCP (5 mg/kg i.p. b.i.d. for 7 days) or saline (1 ml/kg i.p. b.i.d. for 7 days) at approximately 8:00 am and 6:00 pm. 7-28 days of washout was allowed after the completion of PCP administration prior to behavioral testing. D-amino acid oxidase inhibitors or D-serine (100 mg/kg) were dosed 60 minutes prior to the first discrimination problem presented.
Behavioral testing. Behavioral testing was performed according to a modified version of the protocol described by Birrell & Brown. Rats were first habituated to test pots filled with cage bedding and food rewards (Honey Loop cereal), then trained to distinguish which of two scented pots presented contained food reward based on non-spatial cues (digging medium and odor). Finally a series of six discrimination problems were presented; simple discrimination (SD), compound discrimination (CD), intradimensional discrimination (ID), intradimensional reversal discrimination (IDR), extradimensional discrimination (ED) and extradimensional reversal (EDR). Rats only progressed from one discrimination problem to the next (always presented in the same order) after reaching a criterion performance level of six consecutive correct responses.
Statistical analysis. The primary endpoint was the number of trials required to achieve six consecutive correct responses. AU data were analyzed using analysis of variance (ANOVA) techniques. Log ₁₀ transformations of all responses were taken prior to analysis to meet the basic assumptions of homogeneity of variance and normality. A repeated measures ANOVA was performed using rat as a random effect. Rule (discrimination type) was assessed using the within animal variation whilst treatment and starting dimension were assessed using the between animal variation. Interactions between factors were also investigated using the appropriate error term. Specific comparisons between the least squares means for ID and ED rules for each treatment were investigated using orthogonal contrasts and least significant differences (LSDs). Similarly, comparisons between the PCP vehicle group and each of the treatments for each rule were investigated No adjustments were made for multiple comparisons. A typical result is given in Table 1

1-14. (canceled)

15. A compound of the formula (I): 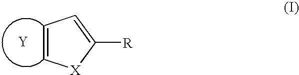
wherein:
R is a carboxylic acid or a salt, ester, anhydride or amide thereof or a hydroxamic acid or a salt thereof,
X is oxygen, sulphur, or NR¹, wherein:
R¹is hydrogen, C₁₋₆alkylcarbonyl which is unsubstituted or substituted with one or two amino groups; or
R¹ is a group —S(O)ₙ—R², wherein R² is amino or C₁₋₆alky which is unsubstituted or substituted with phenyl and n is 0, 1 or 2; or
R¹ is C₁₋₆alkyl which is unsubstituted or substituted with halo or hydroxyl; or
R¹ is (CH₂)ₘAr, wherein m is 0, 1 or 2 and Ar is a five- or six-membered aromatic ring which may be carbocyclic or heterocyclic, which in turn may be substituted by halo, hydroxyl, S(O)ₙ—R², or Ar may be substituted by C₁₋₆alkyl, C₁₋₆alkoxy or fluoro-substituted C₁₋₆alkyl or C₁₋₆alkoxy;
Y is a five-membered heteroaromatic ring containing at least one hetero atom selected from oxygen, nitrogen and sulphur, which ring may be substituted by one or two substituents which are independently selected from:
—S(O)ₙ—R¹, wherein R¹ is amino or C₁₋₆alkyl which is unsubstituted or substituted with phenyl and n is 0, 1 or 2;
hydroxyl, halo, amino optionally substituted by one or two C₁₋₆alkyl groups or C₁₋₆alkyl which is unsubstituted or substituted with hydroxyl, halo or amino, which is unsubstituted or substituted with one or two C₁₋₆alkyl groups; or
the ring Y is unsubstituted or substituted with (CH₂)ₘ¹Ar, wherein ml is 0, 1 or 2, and wherein Ar is unsubstituted or substituted with halo, hydroxyl, amino which is unsubstituted or substituted with one or two C₁₋₆alkyl groups, or
Ar is substituted by C₁₋₆alkyl which is unsubstituted or substituted with hydroxyl, halo or amino which is unsubstituted or substituted with one or two C₁₋₆alkyl groups; or
Ar is unsubstituted or substituted with S(O)ₙR², wherein R² is amino or C₁₋₆alkyl which is unsubstituted or substituted with phenyl and n is 0, 1 or 2.

16. The compound of claim 15 wherein Ar is phenyl, pyridyl or thiazolyl.

17. The compound of claim 15 wherein Y is a five-membered heteroaromatic ring containing one oxygen or sulphur atom, and the ring is unsubstituted or substituted with halo, hydroxyl, methyl or trifluoromethyl.

18. The compound of claim 15 of the formula (II): 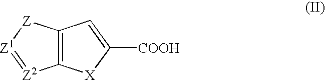
or a salt, ester or anhydride thereof,
wherein:
X is oxygen, sulphur, or a group NR¹ wherein:
R¹ is hydrogen, C₁₋₆alkyl which is unsubstituted or substituted with halo, hydroxyl or
R¹ is a group (CH₂)ₘAr, wherein m is 0, 1 or 2, and Ar is unsubstituted or substituted with halo, hydroxyl, S(O)ₙR², wherein R² is amino, C₁₋₆-alkyl which is unsubstituted or substituted with phenyl and n is 0, 1 or 2; or Ar which is unsubstituted or substituted with C₁₋₆-alkyl, C₁₋₆-alkoxy or fluoro-substituted C₁₋₆alkyl or C₁₋₆alkoxy;
Z is an oxygen or sulphur atom or a group NR¹;
one of Z¹ and Z² is CR³ or N, and the other is CR³, wherein R³ is hydrogen or halo.

19. The compound of claim 15 of the formula (III): 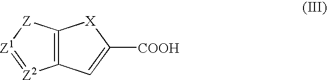
or a salt, ester or anhydride thereof,
wherein:
X is oxygen, sulphur, or a group NR¹ wherein:
R¹ is hydrogen, C₁₋₆alkyl which is unsubstituted or substituted with halo, hydroxyl or
R¹ is a group (CH₂)ₘAr, wherein m is 0, 1 or 2 and Ar is unsubstituted or substituted with halo, hydroxyl, S(O)ₙR², wherein R² is amino, C₁₋₆alkyl which is unsubstituted or substituted with phenyl and n is 0, 1 or 2; or Ar which is unsubstituted or substituted with C₁₋₆alkyl, C₁₋₆alkoxy or fluoro-substituted C₁₋₆alkyl or C₁₋₆alkoxy;
Z is an oxygen or sulphur atom or a group NR¹;
one of Z¹ and Z² is CR³ or N and the other is CR³, wherein R³ is hydrogen or halo.

20. The compound of claim 15 wherein Z is an oxygen or sulphur atom.

21. The compound of claim 15 wherein R³ is hydrogen.

22. A compound which is selected from the group consisting of:
4H-Thieno[3,2-b]pyrrole-5-carboxylic acid;
6H-Thieno[2,3-b]pyrrole-5-carboxylic acid;
3-Bromo-4H-furo[3,2-b]pyrrole-5-carboxylic acid;
4H-Furo[3,2-b]pyrrole-5-carboxylic acid; and
3-Chloro-4H-furo[3,2-b]pyrrole-5-carboxylic acid.

23. A pharmaceutical composition comprising the compound of claim 15 or a pharmaceutically acceptable salt thereof and a pharmaceutically acceptable carrier.

24. A method for the treatment of a subject suffering from a D-amino acid oxidase mediated disease, which comprises administering to that patient a therapeutically effective amount of the compound of claim 15 or a pharmaceutically acceptable salt thereof
